(19) United States
(12) Patent Application Publication
(10) Pub. No.: US 2006/0234391 A1
(43) Pub. Date: Oct. 19, 2006

(54) Optical sensor based on resonant porous silicon structures

(75) Inventors: Sharon M. Weiss, Nashville, TN (US); Jarkko J. Saarinen, Lappeenranta (FI); Philippe Fauchet, Pittsford, NY (US); John Sipe, Toronto (CA)

Correspondence Address: BLANK ROME LLP, 600 NEW HAMPSHIRE AVENUE, N.W., WASHINGTON, DC, 20037, US

(73) Assignee: University of Rochester

(21) Appl. No.: 11/354,831

(22) Filed: Feb. 16, 2006

Related U.S. Application Data
(60) Provisional application No. 60/654,005, filed on Feb. 18, 2005.

(51) Int. Cl. G01N 33/543; C12M 1/34

(52) U.S. Cl. 436/518; 435/287.2

(57) ABSTRACT

A sensor includes at least one high refractive index layer; and at least one low refractive index layer coupled to the high refractive index layer.

### REFERENCE TO RELATED APPLICATION

This application is based on and claims priority under 35 U.S.C. § 119 to U.S. Provisional Patent Application Ser. No. 60/654,005, filed Feb. 18, 2005, the entire contents of which are incorporated herein by reference.

### FIELD OF THE INVENTION

The present invention is directed to a method, system, and apparatus for detecting and identifying target species using a porous silicon waveguide.

### DESCRIPTION OF RELATED ART

Traditionally, analysis of complex refractive indices of gases and absorbing liquids relies on measurements based on the utilization of a prism reflectometer. However, the sensitivity of the system may improve when one side of the prism is coated with a thin metal film to excite a surface plasmon resonance (SPR). SPR devices provide optical sensing technology capable of detecting changes in the refractive indices of gases and liquids.

According to Frost & Sullivan's “World BioChip Market” report of 2001, the worldwide revenue for the broad category of biochips was $1031 million, with a forecasted growth rate of about 60% annually. SPR instruments are the most commonly used devices for protein detection, along with mass spectrometers. The protein array market is expected to grow from $41 million in 2001 at a 55% annual growth rate to $665 million in 2007. In 2007 the instrument business is expected to be about $450 million. The protein array consumables, of which the porous silicon waveguide sensor of the present invention may be included, is projected to be approximately $200 million.

By way of explanation, one side of a high index glass prism may be coated with a thin metal film (e.g., silver or gold) in an SPR sensor. Excitation through the prism creates evanescent fields that extend to the metal-sample interface. Additionally, plasmons on the interface surface may be excited when the component of the incident field wave vector parallel to the surface matches the surface plasmon wave number. The reflectance is decreased when this SPR condition is met, due to absorption in the metal.

The width and depth of the resonance is also changed when any material is placed above the metal. However, this method of detection may be sensitive to unwanted surface defects, such as oxidation of the metal film, since the field strength is largest near the surface and the penetration depth in the sample is relatively small.

Nonetheless, the width of the reflectance dip and the level of the reflectance minimum yield information related to the absorption of the sample and the accuracy of the SPR sensor can be enhanced when the thickness of the metal film is optimized. In this way, SPR can be used for the retrieval of the complex refractive index of any surrounding medium, as well as for the detection of the molecules adsorbed to the metal film. Furthermore, by adding a thin adsorptive layer at the top of the metal film, SPR devices become very sensitive probes for the kinetics of biological binding processes. SPR sensing may also be used to study protein interactions in low-gravity environments.

The development of optical sensors for the detection of chemical and biological species is very important for disease detection, food safety analysis, and biowarfare agent recognition.

### SUMMARY OF THE INVENTION

A first non-limiting aspect of the present invention provides a sensor that includes: at least one high refractive index layer; and at least one low refractive index layer coupled to the high refractive index layer.

A second non-limiting aspect of the present invention provides a method of detecting at least one target species, the method including: binding the at least one target species to a waveguide layer; coupling light through the waveguide layer, the waveguide layer including at least one porous silicon layer having greater porosity than at least one second porous silicon layer; coupling light through the at least one second porous silicon layer; and identifying the at least one target species based on at least one of the coupling steps.

Yet another non-limiting aspect of the present invention provides a system for detecting at least one target species, the system including: means for binding the at least one target species to a waveguide layer; means for coupling light through the waveguide layer; and means for identifying the at least one target species.

The following references may describe additional information related to certain aspects of the present invention, and are incorporated herein by reference in their entireties:

R. L. Rich and D. G. Myszka, “Survey of the 1999 Surface Plasmon Resonance Biosensor Literature,” J. Mol. Recognit. 13, 388-407 (2000);

E. Kretschmann, “Decay of Non-Radiative Surface Plasmons into Light on Rough Silver Films. Comparison of Experimental and Theoretical Results,” Opt. Comm. 6, 185-187 (1972);

V.S.-Y. Lin, K. Motesharei, K. P. S. Dancil, M. J. Sailor, and M. R. Ghadiri, “A Porous Silicon-Based Optical Interferometric Biosensor,” Science 278, 840-843 (1997);

S. Chan S, S. R. Homer, P. M. Fauchet, and B. L. Miller, “Identification of Gram Negative Bacteria Using Nanoscale Silicon Microcavities,” J. Am. Chem. Soc. 123, 11797-11798 (2001);

S. M. Weiss, H. Ouyang, J. Zhang, and P. M. Fauchet, “Electrical and Thermal Modulation of Silicon Photonic Bandgap Microcavities Containing Liquid Crystals,” Optics Express 13, 1090-1097 (2005).

S. M. Weiss and P. M. Fauchet, “Electrically Tunable Porous Silicon Active Mirrors,” Phys. Stat. Sol. A 197, 556-560 (2003).

L. Canham, ed. Properties of Porous Silicon (INSPEC, London, UK, 1997);

V. Lehmann, Electrochemistry of Silicon: Instrumentation, Science, Materials and Applications (Wiley-VCH, Weinheim, Germany, 2002);

W. Theiβ, “Optical Properties of Porous Silicon,” Surf. Sci. Rep. 29, 91-192 (1997);

J. J. Saarinen, J. E. Sipe, S. M. Weiss, and P. M. Fauchet, “Optical Sensors Based on Resonant Porous Silicon Structures,” Optics Express 13, 3754-3764 (2005);

G. Amato, L. Boarino, S. Borini, and A. M. Rossi, “Hybrid Approach to Porous Silicon Integrated Waveguides,” Phys. Stat. Sol. (a) 182, 425-430 (2000);

J. von Behren, L. Tsybeskov, and P. M. Fauchet, “Preparation, Properties and Applications of Free-Standing Porous Silicon Films,” in Microcrystalline and Nanocrystalline Semiconductors, vol. 358, R. W. Collins, C. C. Tsai, M. Hirose, F. Koch, and L. Brus, eds. (Mat. Res. Proc., 1995), pp. 333-338; and

J. J. Saarinen, S. M. Weiss, P. M. Fauchet, and J. E. Sipe, “Reflectance Analysis of a Multilayer 1-D Porous Silicon Structure: Theory and Experiment,” submitted to J. Appl. Phys.

### BRIEF DESCRIPTION OF THE DRAWINGS

In the following description, like reference numerals refer to like elements throughout, wherein:

FIG. 1(a) illustrates a non-limiting example of a method of porous silicon formation;

FIG. 1(b) illustrates a non-limiting example of a relationship between applied current density and porous silicon porosity;

FIGS. 2(a) and 2(b) illustrate non-limiting examples of the impacts of pore size and density on the refractive index of porous silicon;

FIGS. 3(a) and 3(b) include exemplary images of porous silicon with different porosities and different pore sizes;

FIGS. 4(a) and 4(b) present certain non-limiting features related to the performance of a sensor according to a non-limiting example of the present invention;

FIG. 5 illustrates a non-limiting example of a configuration of a sensor according to the present invention;

FIG. 6 presents an exemplary relationship between performance, thickness, and porosity;

FIG. 7 presents a non-limiting example of the relationship between pore morphology and refractive index;

FIG. 8 illustrates a non-limiting example of a target-dependent optimal pore size;

FIG. 9 presents an illustration of the exemplary dependence of apparent pore size on location of cleavage;

FIG. 10 presents a non-limiting example of the functionality of the porous silicon according to an aspect of the present invention;

FIG. 11(a) illustrates a non-limiting example of an SPR sensor;

FIG. 11(b) illustrates a non-limiting example of a waveguide layer according to the present invention;

FIG. 12 provides a non-limiting comparison between the exact SPR reflectance and the SPR reflectance obtained form the pole expansion;

FIG. 13 illustrates a numerical solution of a dispersion relation for κₘ as a function of the waveguide thickness d;

FIG. 14(a) illustrates an example of reflectance as a function of the angle of incidence without nanoparticles;

FIG. 14(b) illustrates a non-limiting example of reflectance as a function of the angle of incidence with nanoparticles; and

FIGS. 15(a) and 15(b) illustrate reflectance from an SPR sensor as a function of the angle of incidence in vacuum and with 1.44 nm thick polymer film on top of a metal film, respectively.

### BRIEF DESCRIPTION OF THE PREFERRED EMBODIMENT

Non-limiting exemplary embodiments of the invention will be set forth in detail with reference to the drawings, in which like reference numerals refer to like elements or steps throughout. In the present invention, porous silicon waveguides may lead to a change in refractive index and measurable deviation in the angle at which light exits the waveguide.

In one non-limiting configuration, one side of a high index glass prism may be coated with a thin metal film onto which specific analytes or ligands may be immobilized. When light is incident on the opposite side of the prism at a desired angle, an evanescent field extending to the metal surface can excite a surface plasmon, or collective oscillation of free electrons propagating along the metal surface.

At certain incidence angles, a resonance may appear in the reflectance signal based on absorption due to the presence of the surface plasmon. The resonance angle may depend on the local refractive index near the metal surface.

Biomarker identification may be performed by monitoring the resonance angle as biomolecules are exposed to the functionalized metal surface. Selective binding causes a change in refractive index and a shift in the resonance angle. The sensitivity of the measurement is then based on the width and depth of the resonance in addition to the magnitude of the resonance shift in response to the binding.

However, two main drawbacks may occur with this configuration. These drawbacks result in large part because the electric field strength may be largest near the surface and may decay exponentially as a function of distance from the metal film.

The first of these drawbacks is that SPR sensors are sensitive to unwanted surface defects. This, in turn, may lead to false positives. Second, the penetration depth of the field in the biomolecules may be small, which may compromise the ultimate sensitivity of the sensor to a given refractive index change.

In light of these drawbacks, an alternative configuration may include a concentrated electric field inside the waveguide where the biological molecules are concentrated. As a result, the infiltration of biomolecules may be improved and a smaller volume of analyte may be required. Additionally, CMOS-compatible devices are now possible for portable detection systems.

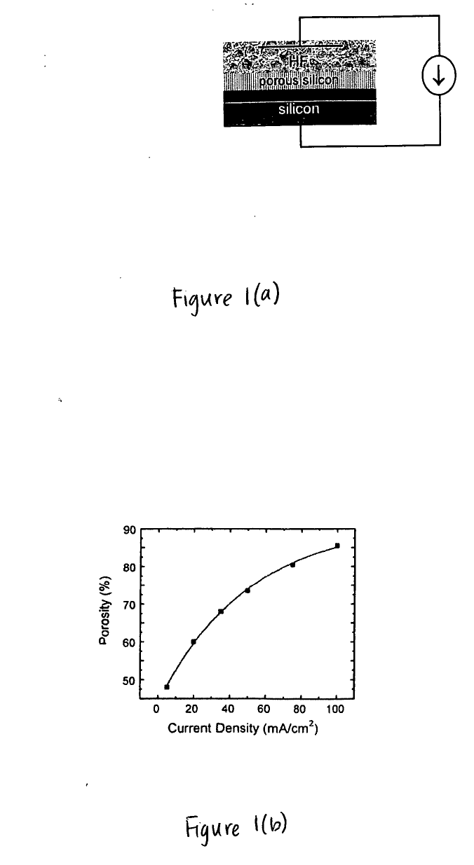

To achieve the concentrated electric field inside the waveguide, a non-limiting aspect of the present invention provides a porous silicon structure. By way of explanation, porous silicon structures may include a network of air holes in a silicon matrix. One method of porous silicon formation may include electrochemical etching in a hydrofluoric acid-based electrolyte, as illustrated in FIG. 1(a). FIG. 1(b) illustrates a non-limiting example of a relationship between applied current density and porous silicon porosity using p+(0.01 Ω-cm) silicon wafers and 15% ethanoic hydrofluoric acid.

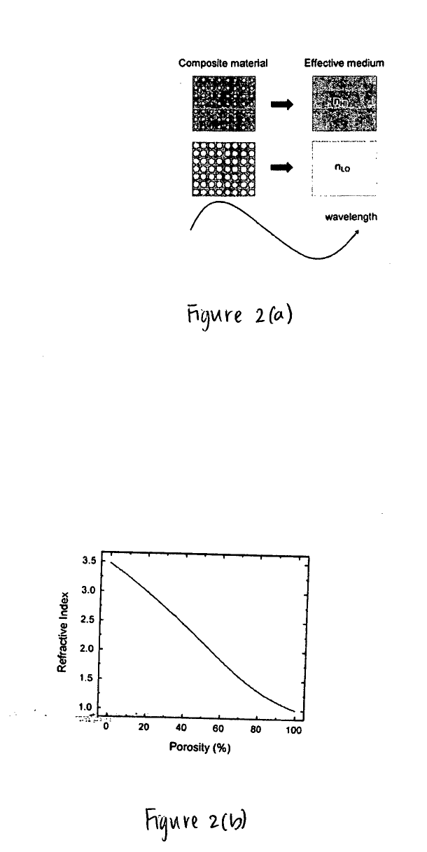

When the pore size is smaller than the wavelength of incident light, porous silicon acts as an effective medium, as illustrated in FIGS. 2(a) and 2(b). In this way, the volume of void space within the silicon matrix (i.e., porosity), which is governed by the pore size and density, determines the refractive index of the porous silicon, as illustrated in FIG. 2(b). A change in applied current density may directly correspond to a change in porous silicon porosity.

The Bruggeman effective medium approximation may be used to express the relationship between porous silicon porosity and refractive index. In practice, for a given silicon substrate and electrolyte, a porosity range of approximately 30% to 80% is attainable. This corresponds to a refractive index range of approximately 1.32 to 2.72 at 1.5 μm. This large refractive index contrast is advantageous for multilayer porous silicon structures with tailored reflectance and transmission spectra that are useful for silicon photonic devices.

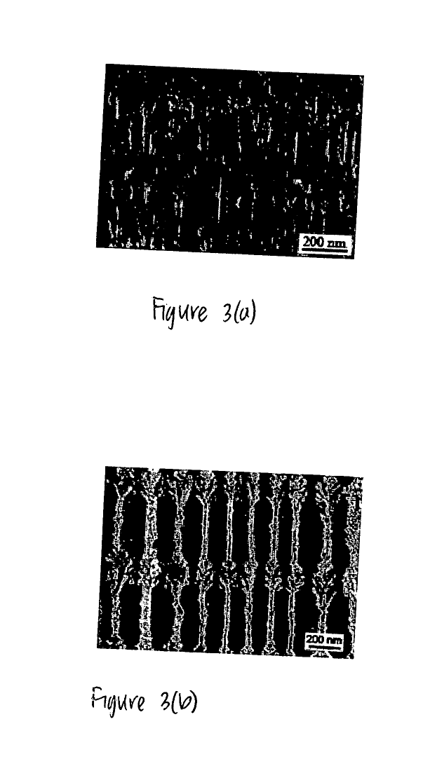

Non-limiting examples of porous structures that may be used for non-limiting aspects of the present invention are illustrated in FIGS. 3(a) and 3(b). Porous silicon formed on p+(0.01 Ω-cm) may be able to support pores having diameters of approximately 5-50 nm. Porous silicon formed on n+(0.01 Ω-cm) may support pore openings of approximately 20-150 nm. The morphology of porous silicon may be tuned from straight pores to branchy pores, with the higher porosity layers tending to have smoother side walls. The thickness of each layer may be determined by the duration of the applied current density walls, but may be limited by the thickness of the silicon substrate. Therefore, it is possible to realize porous silicon waveguides with several degrees of freedom in pore size, shape, and depth that may be used in embodiments of the present invention.

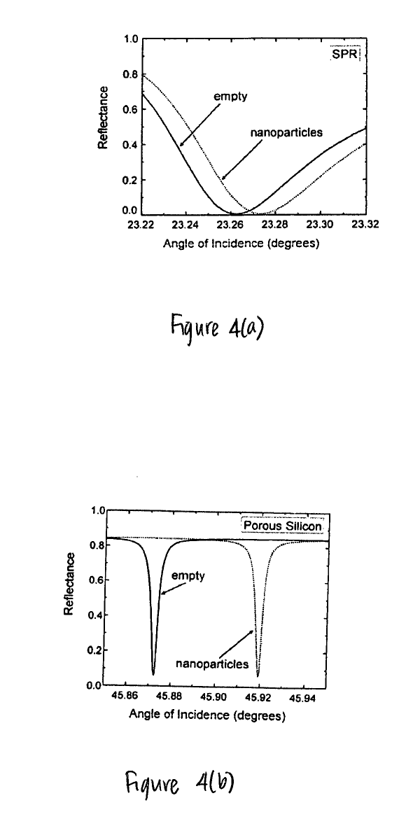

Capabilities of the porous silicon waveguide sensor are illustrated in FIGS. 4(a) and 4(b). As illustrated in FIGS. 4(a) and 4(b), pores of the waveguide layer were theoretically filled with particles of refractive index n=1.59. This porous silicon sensor was then compared to that of a standard SPR sensor with the same amount of nanoparticles spread across the surface of the metal film. The nanoparticles fill 1% of the volume of the pores of the porous silicon waveguide sensor and form a 1.44 nm thick layer on top of the SPR sensor. The porous silicon waveguide sensor resonance has a half-width of 0.004° and the resonance may shift by 0.047° when exposed to the nanoparticles. As shown in FIGS. 4(a) and 4(b), the SPR sensor resonance has a larger half-width of 0.06° and may shift by only 0.011° when exposed to the nanoparticles.

As further illustrated in FIGS. 4(a) and 4(b), the performance of the porous silicon waveguide sensor exceeds the performance of the traditional SPR sensor. Using the shift in the angular position of the resonance divided by the resonance half-width as a measure of performance, the porous silicon waveguide sensor shows a sixty-fold improvement in sensitivity as compared to the SPR sensor.

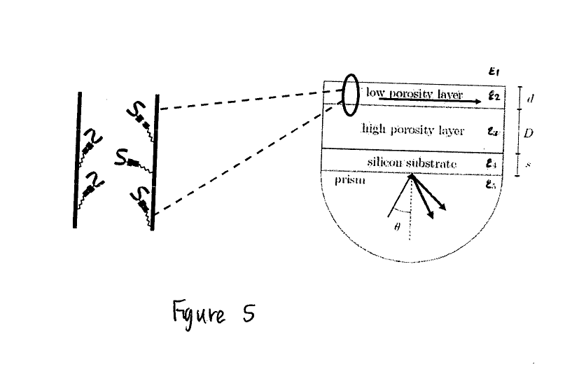

A non-limiting example of a porous silicon waveguide according to the first non-limiting embodiment of the present invention is illustrated in FIG. 5. As shown in FIG. 5, light incident on a prism, such as a rutile prism, may be evanescently coupled through the silicon substrate and high porosity layer to the low porosity waveguiding layer. Light may be confined in the low porosity (high refractive index layer) by total internal reflection, since the low porosity layer is surrounded by air above and low refractive index porous silicon below. Light couples into a propagating waveguide at a specific angle that depends on the refractive index of the porous silicon. The measured reflectance spectrum as a function of angle is characterized by a waveguide resonance (see, e.g., FIGS. 4(a) and 4(b)), where the resonance may correspond to the coupling angle. The porous silicon waveguide according to this embodiment may act as an active sensor because molecular binding of chemical or biological species inside the porous silicon may change the refractive index of the waveguide and may alter the resonance angle. As a result, the intensity of reflected light may be altered at a given angle.

According to this non-limiting embodiment of the present invention, the porous silicon waveguide sensor configuration may be similar to that of SPR sensors, since light is coupled into both sensors based on evanescent wave prism coupling. Therefore, instead of using an SPR chip that includes a metal film deposited on a glass substrate, a porous silicon waveguide can be placed inside the SPR instrumentation for testing of target materials.

One advantage of the porous silicon waveguide of the first non-limiting embodiment is that the electric field may be strongest in the location where the biological and chemical species are detected (e.g., inside the waveguide). Consequently, the porous silicon waveguide sensors may be more sensitive to smaller refractive index changes.

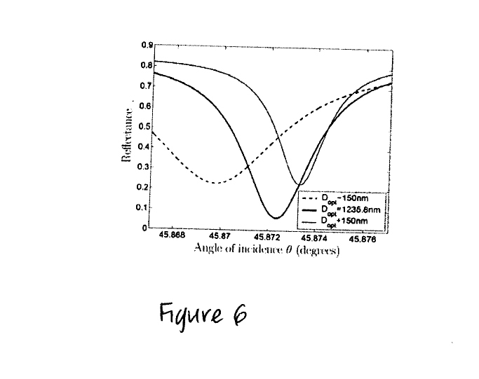

As illustrated in FIG. 6, the performance of the porous silicon waveguide sensor may depend on the porous silicon layer thickness and porosity. The depth and width of the waveguide resonance may be determined by the porous silicon layer thickness, porosity, and absorption losses, among other factors. Generally, the narrower the waveguide resonance, the more sensitive the sensor is to small refractive index changes. Small refractive index changes may cause small angular deviations of the output light. The theoretical analysis shown in FIGS. 4(a) and 4(b) assumes a porous silicon waveguide loss of 10 dB/cm. However, this value may need to be experimentally determined for each porous silicon waveguide configuration before a final specification can be calculated.

As shown in FIG. 6, if the high porosity coupling layer is too thick or too thin, the waveguide resonance may become broader and shallower, which may reduce the sensitivity of the porous silicon waveguide sensor. The propagation loss may depend on porosity, substrate doping, and whether the substrate has been oxidized.

The doping of the silicon substrate may also affect the amount of light coupled into the waveguide. The sensor may operate at 1.5 μm, which is a region of transparency for intrinsic silicon. However, when the porous silicon is formed on highly doped p+ and n+silicon substrates, free carrier absorption may change these characteristics. Using thinner silicon wafers may minimize overall absorption. Since the porous silicon morphology may depend on the substrate doping, the relationship between porous silicon morphology (size and shape of pores) and losses (absorption loss in the silicon substrate and propagation loss in the porous silicon waveguide, for example) may also be factors.

While minimizing the losses, it may sometimes be important to prevent the pore size from becoming too large. If the pore size becomes too large, the effective medium approximation may no longer be valid. On the other hand, if the pore size becomes too small, the target species may have difficulty adsorbing or may not be able to adsorb inside the porous silicon. As an alternative, it may be possible to partially or entirely remove the silicon substrate after porous silicon formation by standard electrochemical techniques. However, removing the silicon substrate, while improving the absorption losses, may compromise the robustness of the waveguide sensor.

A second non-limiting embodiment of the present invention may include coupling light into the porous silicon waveguide from the top surface. Through this configuration, it is possible to reduce substrate absorption. However, this configuration may limit the flexibility of the sensor for measuring opaque target material. Light that is unable to pass through an opaque sample may not couple onto the waveguide to create resonance in the optical spectrum. Nevertheless, for measuring small refractive index changes in transparent material, the configuration of the second embodiment may be useful.

As explained above, the size and shape of the porous silicon features may impact the refractive index, confinement of the optical waveguide mode, concentration of the electric field, and the ability to infiltrate chemical and biological species. An effective medium approximation may be used to relate the porosity (percentage of void space) to a wavelength dependent refractive index. The approximation may be valid as long as the feature size is smaller than the wavelength of incident light. Because the sensors of the first two non-limiting embodiments operate at 1.5 μm, pore diameters less than 200 nm may be beneficial.

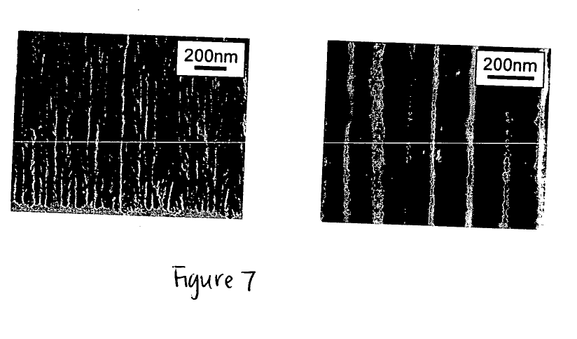

The accuracy of a particular effective medium approximation may also depend on morphology. As shown in FIG. 7, pore morphology may play an important role in determining the porous silicon refractive index and the types of target materials that can be detected using the porous silicon waveguide sensor. For example, the refractive index of a 70% porosity porous silicon layer characterized by branchy, interconnected 20 nm pores is not necessarily the same as that of a 70% porosity porous silicon layer characterized by smooth, isolated 150 nm pores. Therefore, theoretical predictions may only provide guidelines for determining an optical porous silicon waveguide dimension. Fine tuning should generally be accomplished experimentally.

Generally, ultimate sensor performance may be linked to the porous silicon waveguide specifications, since the refractive index and thickness may determine what fraction of the waveguide mode is localized in the low porosity waveguide layer and how intensely the field is concentrated there. Enhancing the interaction between the optical mode, electric field, and target material may lead to the most sensitive sensor devices.

The porous silicon waveguide sensor of the present invention may be used to target a variety of target species, including (as non-limiting examples): DNA, proteins, and toxins. These target species may have sizes less than approximately 150 nm. According to a third non-limiting embodiment of the present invention, the pore size of the waveguide may be adjusted based on the target species. The waveguide of the third embodiment may include the waveguides of the first and second embodiments.

By way of explanation, if the pore size is too small, the molecules will not adsorb inside the porous silicon. However, if the pore size is too large relative to the target species, the effective refractive index change of the porous silicon waveguide may be small, which may reduce the sensitivity of the device.

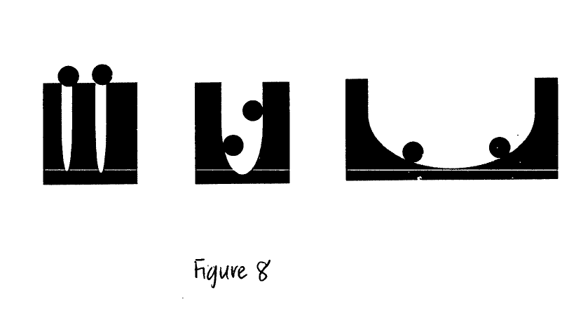

FIG. 8 illustrates a non-limiting example of a target-dependent optimal pore size. As shown in FIG. 8, the pore size of the silicon waveguide may be adjusted to accommodate different sized target species. However, smaller pore sizes may prohibit unwanted large species from infiltrating the active region of the device.

It may be beneficial to compromise between surface area and pore size. Smaller pores tend to be branchy and have larger internal surface areas than the larger pores. Larger pores tend to have smoother side walls. For target species smaller than a few nanometers, branchy pores with narrower pore openings may be preferred. The larger internal surface area may provide more potential binding sites for the target species. However, for larger target species approaching 100 nm in size, smooth pores with large pore openings may be preferred to enable efficient infiltration into the porous silicon waveguide.

To determine the characteristics of the porous silicon morphology, it may be possible to use SEM analysis. This analysis may account for electrochemical etching conditions, electrolyte composition, and substrate doping, which may be varied to develop different porous silicon waveguides.

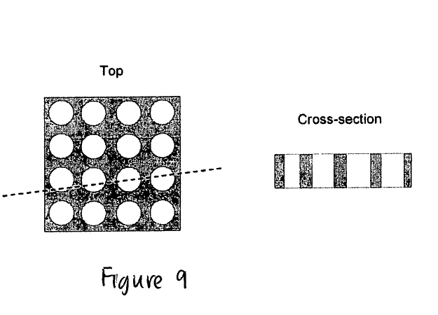

However, the traditional techniques of using SEM and TEM instruments to show the top view and cross-sectional view of porous silicon features may not be as accurate as desired. In more detail, the pore size and density as viewed from the top is not necessarily maintained in depth as the etching reaction does not instantaneously stabilize. Moreover, cross-sectional images do not reveal the true pore diameter and morphology, since the apparent pore opening depends on the location of the cleavage, as shown in FIG. 9. Therefore, to determine a more accurate size and shape of the porous silicon features, it may be beneficial to take several images at various depths in the porous silicon structure. This may be done through software imaging, for example.

According to a third non-limiting embodiment of the present invention, it may be possible to configure the porous silicon waveguide to be sensitive to only a particular target species or biomaterial. The third non-limiting embodiment may include any of the biosensors set forth in the preceding embodiments.

To configure the porous silicon waveguide to be sensitive to a particular target species, it is possible to adjust the surface chemistry. The surface chemistry may be adjusted by attaching probe molecules to the internal porous silicon surface. Ideally, only the target species should bind to the probe to prevent false positives.

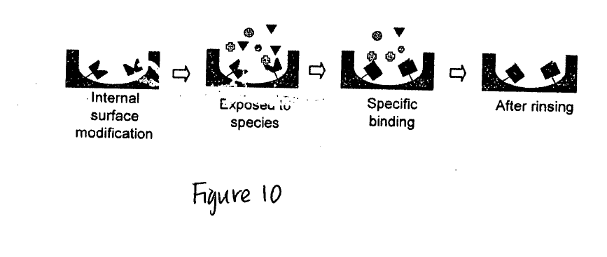

The functionalization of porous silicon is illustrated in FIG. 10. First, the porous silicon is oxidized so that an aminosilane may bind to the internal surface. If the probe cannot bind directly to the silane, another surface linker may be added before the probe is infiltrated into the porous silicon.

Non-specific binding may be prevented in at least two ways. For example, the higher the affinity of the probe-target pair, the less likely non-specific binding will occur. Therefore, it may be desirable to configure highly selective probe for each target molecule. By way of non-limiting example, several antibody-antigen systems naturally have high affinity for each other. For example, the Biotin-Strepdavidin pair may be used for its high affinity.

Additionally, a blocking agent may be used to prevent non-specific binding of the target species. If the target species can bind to the silane or other surface linker, then a blocking agent (such as, for non-limiting example, Bovine Serum Albumin (BSA)) may prevent the target from immobilizing anywhere on the internal surface other than on the probe molecule.

After the target species is exposed to the sensor, the sensor may be rinsed to remove any material that is not bound to the probe molecules, as illustrated in FIG. 10. For integrated systems, the rinsing may be performed using microfluidics or other techniques known to those of skill in the art.

To evaluate where the target molecules reside inside the waveguide, environmental SEM imaging tools may be used. Electron microscopy of liquids may, at times, be difficult, so a highly sensitive instrument may be required. By tagging the target molecules with gold nanoparticles, the SEM images may reveal the exact location of the biological materials inside the porous silicon. The morphology of the porous silicon waveguide sensor may then be optimized for each target material to ensure efficient and uniform infiltration into the sensor.

The target species only need to be present in the low porosity waveguiding layer where the field is focused. Since the optical thickness of the high porosity layer may be configured to efficiently couple light into the waveguide and to confine the optical mode, infiltration of an unknown quantity of target material into that layer may slightly degrade the quality of the waveguide resonance. Furthermore, localizing the target material in the top porous silicon layer may reduce the overall quantity of target material required for detection.

Modifications to the porous silicon waveguide configuration discussed above may include etching a very low porosity, small pore size porous silicon layer of a few nanometer thickness between the low porosity waveguiding and high porosity coupling layers. This intermediate layer may be thin enough to have minimal impact on the optical properties of the device while preventing biomolecules from penetrating into undesired regions.

The porous silicon waveguides of the present invention may have a number of advantages. First, due to the direct interaction of electromagnetic fields and biomaterials at the nanoscale, more reliable detection of biological and chemical species at lower detection limits may be achieved. In other words, the sensor of the present invention is capable of responding to smaller quantities of target material that previously available sensors.

Additionally, using porous silicon as the host material improves flexibility in tailoring the pore size and shape to allow the target species to adsorb into the porous silicon waveguide while unwanted larger species may be filtered out. The porous silicon waveguides may also function as general purpose, label free sensors because the morphology and surface functionalization may be adjusted to detect a variety of medically significant biomarkers from DNA signatures for genetic diseases such as cystic fibrosis to proteins such as the CA125 biomarker for ovarian cancer.

Moreover, the large internal surface area of porous silicon may enable the sensor to have a small footprint, which is especially useful for configurations that relate to CMOS-compatible, integrated sensor systems. The porous silicon waveguide also has a low production cost, so single-use, disposable sensors may be feasible.

The porous silicon waveguide sensors provide excellent models systems for confirming the effect of confining biomaterials into nanoscale domains. By mapping the interaction of electromagnetic fields and biomolecules at size scales from 1-150 nm, the sensitivity and flexibility of the sensor may be improved.

Another non-limiting aspect of the present invention may include prism coupling to excite a waveguide mode. In this way, the porous silicon waveguide of the present invention may be compatible with commercialized SPR instrumentation. In other words, according to this aspect of the present invention, a porous silicon waveguide sensor may be incorporated into the SPR instrument instead of the traditional metal film coated SPR chip.

A fourth non-limiting aspect of the present invention provides a boron-doped silicon substrate in which wavelength absorption is small. Small absorption enables direct coupling through the doped silicon substrate using, for example, a high index glass prism. Therefore, it may be possible to eliminate a process of lifting off thin porous silicon layers from the substrate. However, if a high index glass prism is not included, it is possible to lift off the thin porous silicon layers from the substrate to achieve similar effects.

For operation at visible wavelengths, where absorption tends to be increased, the fourth embodiment may include a lift-off process to position the porous silicon on top of non-absorbing materials. Alternatively, to minimize absorption losses, it is also possible to replace the porous silicon coupling layer with an appropriate layer of a non-absorbing low index material.

For purposes of analysis, it is convenient to split the wave numbers into parallel and perpendicular pieces with respect to the interfaces. Wave number parallel to the surface is denoted by κ, which may be assumed to be real. Hence, the wave number perpendicular to the surface wᵢ is given by:
wᵢ=(\tilde{ω}^{2 ε}ᵢ−κ²)^{1/2} (1)
with the wave number denoted by \tilde{ω}=ω/c and where Eᵢ is the dielectric function of the ith medium. The square root may be defined such that IM{z^{1/2}}≧0 and Re{z^{1/2}}≧0, if Im{z^{1/2}}=0. The reflectance may be analyzed by the Fresnel reflection and transmission coefficients, which in this embodiment are represented by:

$${r}_{ij}^{s} = \frac{{w}_{i} - {w}_{j}}{{w}_{i} + {w}_{j}} ; {t}_{ij}^{s} = \frac{2 {w}_{i}}{{w}_{i} + {w}_{j}} , \qquad ( 2 )$$

for s-polarized light, and by:

$${r}_{ij}^{p} = \frac{{w}_{i} {ɛ}_{j} - {w}_{j} {ɛ}_{i}}{{w}_{i} {ɛ}_{j} + {w}_{j} {ɛ}_{i}} ; {t}_{ij}^{p} = \frac{2 {n}_{i} {n}_{j} {w}_{i}}{{w}_{i} {ɛ}_{j} + {w}_{j} {ɛ}_{i}} . \qquad ( 3 )$$

for p-polarized light. The notation is that the field may be incoming on an interface between media i and j from medium i. The expressions (2) and (3) are also valid for absorbing media, and satisfy the Fresnel coefficient identities:
rᵢⱼ=−rⱼᵢ:tᵢₖtⱼᵢ−rᵢⱼrⱼᵢ=1 (4)
Equations (1)-(4) may be used in the derivation of the pole expansion analysis of the reflectance from the SPR and porous silicon sensors in this non-limiting embodiment.

As explained above, surface plasmons are electromagnetic modes propagating at the interface between the metal film and the sample, and the phenomenon can be described as a collective oscillation of free electrons in the metal layer. These modes are generally excited with p-polarized light, since the incident electric field of the s-polarized light may not include a component of the wave vector parallel to the interface pointing in the direction of surface plasmon propagation along the interface. The dispersion relation for surface plasmons may be obtained when the incident wave number parallel to the interface matches together with the SPR wave number. At this resonance condition, the Fresnel reflection coefficient r₃₁, given by Equation (3) for p-polarized light, diverges. Hence r₃₁ may have a pole at a particular wave number, and this pole signals the dispersion relation for a surface plasmon:

$${κ}_{SPR} = {\tilde{ω} ( \frac{{ɛ}_{1} {ɛ}_{3}}{{ɛ}_{1} + {ɛ}_{3}} )}^{1 / 2} , \qquad ( 5 )$$

where κ_{SPR} is the complex wave number of the surface plasmon, with ε₁ and ε₃ denoting the dielectric function of the sample and the metal, respectively. For ease of analysis, the following description is representative of a single wavelength, which allows the dispersion of the constituent materials to be omitted. Of course, the analysis can be performed for any wavelength, and by superposition could be extended to treat the response of the device to pulsed irradiation, for example.

By expanding r₃₁ in the neighborhood of the pole (5), the reflection coefficient may be expressed as:

$${r}_{31} ≅ \frac{{κ}_{s}}{κ - {κ}_{SPR}} . \qquad ( 6 )$$

The complex pole strength parameter κₛ is:

$${κ}_{s} = - \frac{2 {ɛ}_{1}^{2} {ɛ}_{3}^{2}}{( {ɛ}_{1} + {ɛ}_{3} ) ( {ɛ}_{1}^{2} - {ɛ}_{3}^{2} )} \frac{{\tilde{ω}}^{2}}{{κ}_{SPR}} , \qquad ( 7 )$$

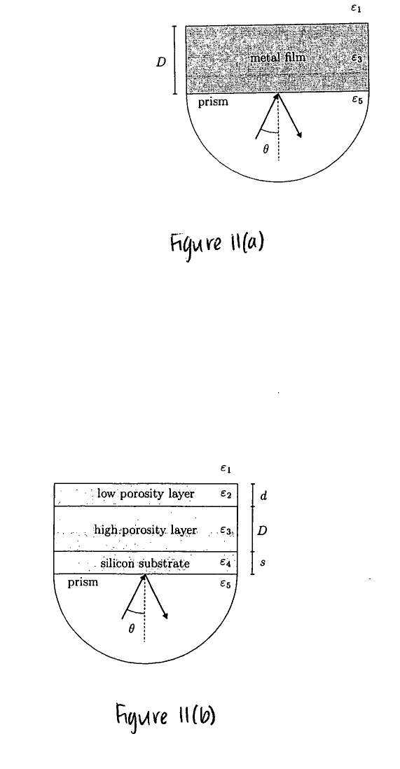

which is a general expression and is valid for absorbing samples (complex valued ε₁) as well. The validity of the pole expansion may be demonstrated by comparing calculated reflectance to the exact one when the SPR sensor is in vacuum. While this example is selected for simplicity, the pole strength parameter can be approximated as real for typical metals (e.g., Ag and Au), whose real part of the dielectric function is larger than the imaginary part (e.g., |Re({ε₁}|>>|Im{ε₃}|). Hence, the pole strength may be approximated by:
κₛ=−4(Re{κ_{SPR}}−\tilde{ω}) (8)
To obtain the SPR reflectance R₅₁, it is possible to consider the squared modulus of complex reflectivity \tilde{r}₅₁. The tilde denotes an effective Fresnel coefficient connecting the two indicated media, where there may be any number of layers of other media in between. For example, referring to FIG. 11(a),

$${\tilde{r}}_{51} = {r}_{53} + \frac{{t}_{53} {r}_{31} {t}_{35} exp ( 2 ⅈ {w}_{3} d )}{1 - {r}_{31} {r}_{35} exp ( 2 ⅈ {w}_{3} d )} = \frac{{r}_{53} + {r}_{31} exp ( 2 ⅈ {w}_{3} d )}{1 - {r}_{31} {r}_{35} exp ( 2 ⅈ {w}_{3} d )} , \qquad ( 9 )$$

where the Fresnel coefficient identify (4) is applied. Equation (9) provides the observed reflectance from the SPR shown in FIG. 11(a). The pole approximation results from using approximation (6) rather than the exact expression for r₃₁.

One advantage of the pole expansion analysis is that analytic expressions (albeit approximate) for the reflectivity \tilde{r}₅₁ are easily obtained and physically interpreted. Reflection coefficient r₅₃ may be expressed as r₃=−r₃₅−=Aexp(−iφ), where both A and φ are real. Thus, this may enter the total internal reflection regime. For highly reflecting metals, A≈1 and |r₅₃|=|r₃₅|=1. The phase of this coefficient in this approximation may be obtained from the expression:

$$ϕ = 2 {tan}^{- 1} ( \frac{Im \{ {w}_{3} \} {ɛ}_{5}}{Re \{ {w}_{5} \} Re \{ {ɛ}_{3} \}} ) \qquad ( 10 )$$

with both w₃ and w₅ calculated at the pole with κ=κ_{SPR}. It may be convenient to separate the real and imaginary parts of the complex pole κ_{SPR} and express r₃₁ as follows:

$${r}_{31} = \frac{{κ}_{s}}{κ - ( {κ}_{m} + i γ )} with {κ}_{m} + i γ = {κ}_{SPR} . \qquad ( 11 )$$

SPR reflectance R₅₁=|\tilde{r}₅₁|² may then be determined by inserting these expressions into Equation (9) as follows:

$$\begin{matrix}\begin{matrix}{R}_{51} = {\vert \frac{exp ( - ⅈϕ ) + β {r}_{31}}{1 + β {r}_{31} exp ( - ⅈϕ )} \vert}^{2} \\ = 1 - \frac{4 β Im \{ {r}_{31} \} sin ϕ}{1 + {β}^{2} {\vert {r}_{31} \vert}^{2} + 2 β ( cos ϕ Re \{ {r}_{31} \} + sin ϕ Im \{ {r}_{31} \} )} , \\ = 1 - \frac{4 {βγκ}_{s} sin ϕ}{{( κ - {κ}_{m} + {βκ}_{s} cos ϕ )}^{2} + {( γ + {βκ}_{s} sin ϕ )}^{2}} ,\end{matrix} & ( 12 )\end{matrix}$$

where the Fabry-Perot phase factor of Equation (9) may be approximated in the vicinity of the pole by β=exp(2iw₃d)≈exp(−2\tilde{ω}\sqrt{|1−ε₃|}d) Incident wavelength is estimated to be λ=1.532 μm, which provides a reasonable comparison between the SPR sensor and a porous silicon sensor of the fourth non-limiting embodiment.

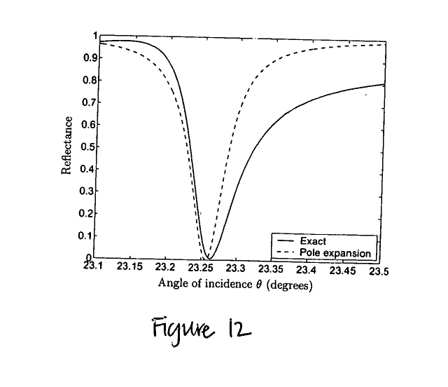

It is possible to calculate the reflectance from a standard SPR device with a silver film (refractive index n₂=0.462+9.20i, thickness d=40 nm) on top of the rutile prism (refractive index n₃=2.55). The comparison between the exact SPR reflectance (solid line) and the SPR reflectance obtained from the pole expansion (dashed line) is illustrated in FIG. 12. The exact curve is calculated using Equation (9) and the pole expansion curve from Equation (12). A good fit is observed close to the pole, near the minimum of the reflectance. The discrepancy between the exact calculation and the pole analysis arises largely because β may vary over the angular spread of the surface plasmon dip, while the pole analysis treats β as a constant.

Of course, this pole expansion is not necessary for analyzing the SPR sensor. Even with the addition of the adsorptive layer, the SPR sensor may be modeled by transfer matrices. However, the above analysis is useful for identifying the parameters κₘ, κₛ, ρ, and φ upon which performance of the SPR device generally depends.

Porous silicon structures may contain air pores perpendicular to the interfaces, and may display birefringence. The different dielectric tensor elements εₓₓ=ε_{yy}=ε^{∥} and ε_{zz}=ε^{⊥}, where z is the direction normal to the interface. For purposes of analysis, the air pores have been assumed to be small relative to the wavelength of light, as they are for many fabrication protocols. Thus, it is possible to treat the porous silicon as an effective medium. The following non-limiting explanation considers s-polarized light, which experiences only ε^{∥}. However, analysis of p-polarized light is also possible, and involves both ε^{∥} and ε^{⊥}.

In the porous silicon sensor of the fourth embodiment, light may be coupled through the silicon substrate using a high index prism to the porous silicon layers. A waveguide mode may be excited evanescently through the low index (high porosity) coupling layer to the high index (low porosity) resonator layer. For the evanescent fields in regions 1 and 3 (illustrated in FIG. 11(b)), the following expression applies: n₂\tilde{ω}>n₁\tilde{ω},n₃\tilde{ω}, and the poles of r₃₁ are associated with the usual dispersion relation for the waveguide modes:

$$tan ( hd ) = \frac{q + p}{h ( 1 - qp / {h}^{2} )} , \qquad ( 13 )$$

where w₁=iq, w₂=h, and w₃=ip, with q,h,pΕR, and where d is the thickness of the waveguide layer. Equation (13) is an implicit equation for K as a function of the resonator layer thickness d. The waveguide modes, labeled by κₘ, are the solutions of this equation. These wave numbers are real, as silicon may be assumed to be non-absorbing at the frequency of interest.

The pole expansion of the reflectivity \tilde{r}₃₁ from the waveguide layer in FIG. 11(b) is given by Equation (9) with the replacements 3→2 and 5→3. The pole expansion takes the form:

$${\tilde{r}}_{31} ≅ \frac{{κ}_{s}}{κ - {κ}_{m}} , \qquad ( 14 )$$

where the real valued pole strength parameter κₛ is found to be:

$${κ}_{s} = \frac{2 {p}_{m} {h}_{m}^{2}}{{κ}_{m} {\tilde{ω}}^{2}} \frac{1}{{ɛ}_{1} - {ɛ}_{2}} \frac{{q}_{m} {p}_{m}}{{q}_{m} + {p}_{m} + {q}_{m} {p}_{m} d} , \qquad ( 15 )$$

and all parameters with index m are calculated at the pole, using the value κ=κₘ. As an example, the same incident wavelength (λ=1.532 μm) may be used as was used for SPR calculations. The refractive index of doped silicon is similar to that as for undoped silicon (n_{Si}=3.4784), and absorption is not taken into account in the thin porous silicon layers considered in this example. The porosities of the resonator and coupling layers are chosen to be 50% and 75%, respectively. However, other porosities are within the scope of the present invention, and these porosities are merely non-limiting examples. From a Maxwell Garnett (MG) effective medium theory for cylinders, the following indices may be obtained for the resonator and coupling layers:
n_{Res}^{∥}=2.213, n_{Res}^{⊥}=2.559 and n_{Cp1}^{|}=1.642, and n_{Cp1}^{⊥}=1.943

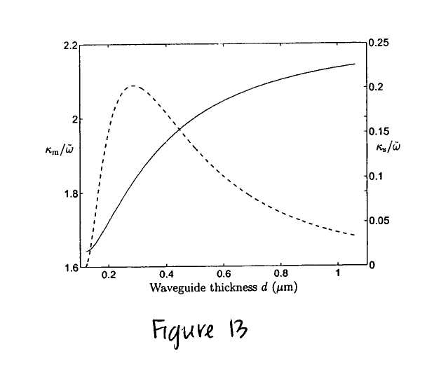

The numerical solution of the dispersion relation for κₘ as a function of the waveguide thickness d is illustrated in FIG. 13. A cutoff value may be observed in the thickness of the waveguide that can support waveguide modes in the asymmetric waveguide, which may be expected. The pole strength, as illustrated in FIG. 13, displays a maximum at d=287.2 nm.

In the foregoing examples, \tilde{r}₃₁ has been considered to be purely real. In that case, all incident light in the geometry of FIG. 11(b) would be reflected, since no energy would be dissipated in the structure. In the evanescent region of interest, no incident light could propagate into medium 1. However, scattering losses may occur as a result of the pores in the porous silicon layers.

As a first approximation, the scattering losses in the coupling layer may be neglected, since the electric field is concentrated in the waveguide layer at resonance. The following explanation accounts for a phenomenological scattering parameter γ to account for losses in the resonator layer. Adjusting the pole expansion in Equation (14) yields:

$${\tilde{r}}_{31} ≅ \frac{{κ}_{s}}{κ - ( {κ}_{m} + i γ )} , \qquad ( 16 )$$

where the value of γ may be determined experimentally.

Thus, the pole κₘ is now shifted to a complex wave number κₘ+iγ in analogy to the pole κ_{SPR} in the SPR pole expansion. This illustrates another strength of the pole analysis, as the approach gives a simple phenomenological treatment of scattering. Of course, scattering losses may be phenomenologically included using the transfer matrix approach by adding an imaginary part to the dielectric function of the waveguide layer. However, the pole expansion provides analytical expressions for the optimization of the structure. Additionally, γ may be set immediately from the experimentally determined waveguide loss. For porous silicon structures (such as those in the non-limiting fourth embodiment of the present invention), loss may range from approximately 1-90 dB/cm.

The observed reflectance R₅₁ from the porous silicon sensor may now be calculated from the usual combinations of Fresnel coefficients that appear in thin film optics. For the exemplary configuration illustrated in FIG. 11(b), while accounting for the scattering described above, R₅₁=|\tilde{r}₃₁|², where:

$$\begin{matrix}{\tilde{r}}_{51} = {r}_{54} + \frac{{t}_{54} {\tilde{r}}_{41} {t}_{45} exp ( ⅈ2 {w}_{4} s )}{1 - {r}_{45} {r}_{41} exp ( ⅈ 2 {w}_{4} s )} & ( 17 ) \\ with & \\ {\tilde{r}}_{41} = \frac{{r}_{43} + {{\tilde{r}}^{′}}_{31} exp ( ⅈ2 {w}_{3} D )}{1 - {r}_{34} {{\tilde{r}}^{′}}_{31} exp ( ⅈ 2 {w}_{3} D )} . & ( 18 )\end{matrix}$$

However, the silicon substrate can be thick, and fluctuations in its thickness over the beam size typically wash out the Fabry-Perot oscillations described by the factors exp(i2w₄s) in Equation (17). Therefore, the denominator in the second term of Equation (17) may be set to unity, and the interference between the two terms may be neglected. This yields:
R₅₁=|r₅₄|²+|1−r₅₄²|²|exp(i2w₄s)|²R₄₁ (19)
where R₄₁=|\tilde{r}₄₁|². The adsorption in the silicon substrate (thickness s=500 μm) may be estimated from the doping level, which may be less than 10¹⁸ B atoms per cm³. It is possible to estimate a value α=1 cm⁻¹ for the absorption, which corresponds to a value 1.22×10⁻⁵ for the extinction coefficient in the doped silicon substrate.

Since at angles of incidence of interest the fields are propagating in medium 4 and evanescent in region 3, r₄₃=−r₃₄=exp(−iφ), and φ may be approximated by its value at the waveguide resonance κₘ:

$$ϕ = 2 {{tan}^{- 1} ( \frac{{κ}_{m}^{2} - {\tilde{ω}}^{2} {ɛ}_{3}}{{\tilde{ω}}^{2} {ɛ}_{4} - {κ}_{m}^{2}} )}^{1 / 2} . \qquad ( 20 )$$

Purely imaginary W₃ may be approximated by its value at the resonance, w₃−ipₘ, and put exp(i2w₃D)≈β=exp(−2pₘD). Then:

$${R}_{41} = {\vert \frac{exp ( - ⅈ ϕ ) + β {\tilde{r}}_{31}^{′}}{1 + β {\tilde{r}}_{31}^{′} exp ( - ⅈϕ )} \vert}^{2} . \qquad ( 21 )$$

Equation (21) is analogous to the expansion Equation (12) used for the SPR reflectance. Hence, the expansion of the reflectance R₄₁ is exactly the same as the first one of Equation (12), except that \tilde{r}′₃₁ is used instead of r₃₁. Using Equation (19), the following equation may be derived:

$$\begin{matrix}{R}_{51} = {\vert {r}_{54} \vert}^{2} + \\ {\vert 1 - {r}_{54}^{2} \vert}^{2} {\vert exp ( ⅈ 2 {w}_{4} s ) \vert}^{2} \\ [ 1 - \frac{4 {βγκ}_{s} sin ϕ}{{( κ - {κ}_{m} + {βκ}_{s} cos ϕ )}^{2} + {( γ + {βκ}_{s} sin ϕ )}^{2}} ] ,\end{matrix} \qquad ( 22 )$$

cf. Equation (12). The minimum reflectivity may be obtained when κ=κₘ−βκₛ cos φ, which corresponds to the critical angle of incidence θ_{crit}. In silicon substrates (κ=n₄\tilde{ω} sin θ), this angle may be given by:

$${θ}_{crit} = {sin}^{- 1} ( \frac{{κ}_{m} - {βκ}_{s} cos ϕ}{{n}_{4} \tilde{ω}} ) \qquad ( 23 )$$

and the corresponding critical angle of incidence in the rutile prism is calculated from Snell's law. At this particular angle, the minimum reflectivity may be:

$${[ {R}_{41} ]}_{min} = {[ \frac{γ - {βκ}_{s} sin ϕ}{γ + {βκ}_{s} sin ϕ} ]}^{2} , \qquad ( 24 )$$

which goes to zero if γ=ρκₛ sin φ. This condition corresponds to a situation where all of the incident light is coupled into a waveguide mode and absorbed by the scattering losses. The optical coupler thickness Dₒₚₜ may be obtained using the definition of β=exp(−2pₘD), in the form:

$${D}_{opt} = - \frac{1}{2 {p}_{m}} ln [ \frac{γ}{{κ}_{s} sin ϕ} ] . \qquad ( 25 )$$

The reflectance from the porous silicon sensor may go to a minimum value at the critical angle with this exemplary coupler thickness. As a result, it may be possible to optimize the configuration of the sensor by choosing this value of D. The thickness of the resonator layer may be chosen arbitrarily, but good sensitivity of the sensor may be obtained where the pole strength is largest. The minimum value and the width of the reflectance dip may be larger for smaller coupler thicknesses as the light may be coupled too efficiently back to the silicon substrate. On the other hand, the excitation of the waveguide mode may be reduced if the coupling laser is thicker than the optimal value, leading to an increase in the minimum value of the reflectance.

The performance of the sensor of the non-limiting fourth embodiment may be demonstrated a sample calculation. In this non-limiting example, a rutile prism (n=2.55) may be placed below the silicon substrate to facilitate optical coupling into the waveguide resonance. The pole expansion, developed in the previous section (which may be limited to angles of incidence for which fields are evanescent in both the coupler and air but not in the resonator) may be applied and scattering losses may be accounted for by adding the appropriate imaginary part y to the resonance wave number associated with the waveguide mode. This example includes y=115.13 m⁻¹, corresponding to a waveguide loss of 10 dB/cm. This is well within the range of losses observed for similar configurations. A resonator layer thickness may be d=287.2 nm, which corresponds to the maximum value of the pole strength curve presented in FIG. 13. The optimal thickness of the coupling layer is found to be D=1235.8 nm from Equation (25).

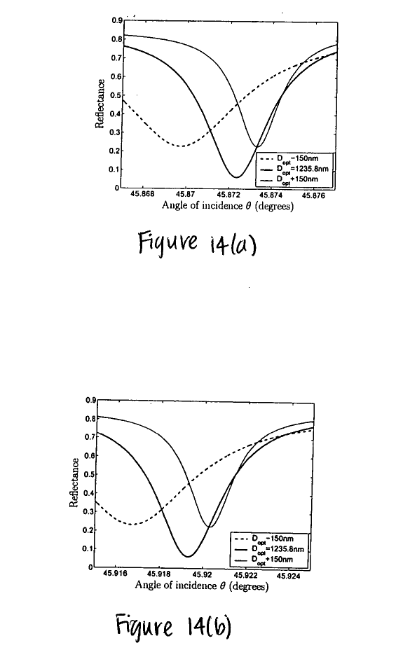

From an experimental point of view, the etching of 290 nm low-porosity layer on top of 1.24 micron high porosity layer on silicon substrate is straightforward. Furthermore, the device operation is not very sensitive to the thickness of the coupling layer, as may be seen from the results displayed in FIGS. 14(a) and 14(b). For larger values of D (thin solid lines) the system is undercoupled and smaller reflectivity dips are exhibited, while for smaller D (dashed lines), the dips become broader as the light may be coupled too efficiently back to the reflected field.

As a simple model for a detection scenario, the pores of the waveguide layer may be at least partially filled with nanoparticles of refractive index of approximately n=1.59. The reflectance of this configuration may then be compared configuration before filling. Assuming that the nanoparticles fill 1% of the volume of the pores, the new refractive index of these pores may be modeled with the Maxwell Garnett theory for spheres in air. For the resonator layer, the MG effective medium theory for cylinders then yields a new n_{Res}^{|=}2.215. The predicted reflectances before and after filling are shown in FIGS. 14(a) and 14(b), respectively. For the air pores a narrow reflectance dip is obtained at an angle of incidence of 45.872°, while for pores partially filled with nanoparticles the corresponding resonance angle is 45.919°, yielding a shift of ΔΘ=0.047°. This shift is in the detection range as the half-width of the dip, which is approximately 0.004° with optimized coupler thickness.

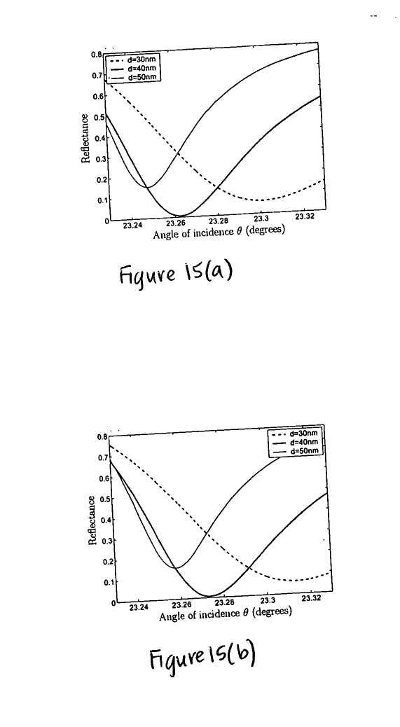

This exemplary porous silicon waveguide may be compared with an SPR device (shown in FIG. 11(a)) by considering silver metal, with refractive index n_{Ag}=0.462+9.20 at λ=1.532 μm on top of a rutile prism. The best sensitivity of the device operation may be observed by taking a metal thickness of d=40 nm, as shown in FIG. 15(a). For larger thicknesses, the reflection minimum increases as the excitation of surface plasmon is reduced due to absorption of the metal film. For smaller thicknesses the surface plasmon may be coupled too strongly back to the reflected field and the reflectance dips become broader as seen in FIGS. 15(a) and 15(b).

In a non-limiting example of an SPR sensor, the target material to be detected may be taken to be on top of the metal film. For the exemplary porous silicon waveguide, the pore volume in a surface area A of the detector was (0.5)(287 nm)A, of which 1% was filled with nanoparticles with n=1.59. The same amount of material per area A here, spread uniformly over the surface of the SPR sensor, would result in an overlayer of thickness (0.01)(287 nm)=1.44 nm. The optical response may be calculated using the transfer matrices, without applying a pole approximation. However, a calculation with the pole approximation would lead to essentially the shifts and widths.

As shown in FIG. 15(a), without the overlayer, the SPR resonance angle of incidence is 23.263°, while the resonance angle is 23.274° with the overlayer. This yields a shift of Δθ=0.011° with a half-width of the SPR resonance dip approximately 0.06° for the optimized metal thickness. Furthermore, this shift is smaller than the predicted shift for the porous silicon sensor, and can be associated with the fact that the target material is exposed only to evanescent fields in the SPR sensor. In the porous silicon sensor, on the other hand, the target material may exist within the volume of the waveguide and may be exposed to the full field of the waveguide mode.

Perhaps more importantly, the dips are significantly broader in the SPR sensor than predicted for the porous silicon sensor. This arises because the scattering loss of the porous silicon sensor waveguide mode, even at 10 dB/cm, is less than the absorption loss of typical surface plasmons; the y for the SPR sensor that follows from Equation (11) corresponds to 214 dB/cm. Any surface roughness or defects near the metal surface would only increase the SPR resonance width.

A reasonable figure of merit with which to compare the devices would be the shift in the position of the resonance dip divided by the width of that dip. For the foregoing example, that figure of merit is approximately 0.20 for the SPR sensor, and 12 for the porous silicon sensor. As a result, the presence of the target material would clearly be much easier to detect with the porous silicon sensor of the non-limiting fourth embodiment. It may also be noted that the operation of the SPR sensor is qualitatively the same if considered at visible wavelengths.

Additional alternative embodiments may include a grating coupled porous silicon waveguide sensor with a laser diode and photodiode array as an integrated device. Ridge waveguides may also be patterned for enhanced lateral electric field confinement and mechanically robust membrane structures can be considered where a window may be opened in the substrate to allow light to reach the waveguide without suffering any absorption. Alternatively, a single layer porous silicon waveguide may be etched on an SOI wafer to eliminate the infiltration of biomaterials beyond the waveguide layer.

Although examples of the present invention have been illustrated and described herein, the scope of the present invention should not be limited to these exemplary embodiments. Accordingly, it will be understood that other configurations and methods may be used that are included in the spirit and scope of the present invention. The present invention, while defined by the following claims, includes all equivalents thereto and should not be considered limited thereby.

We claim:

1. A sensor, comprising:
at least one high refractive index layer; and
at least one low refractive index layer coupled to the high refractive index layer.

2. The sensor according to claim 1, wherein the at least one high refractive index layer includes at least one porous silicon layer.

3. The sensor according to claim 2, wherein the at least one low refractive index layer includes at least one porous silicon layer.

4. The sensor according to claim 3, wherein the at least one low refractive index layer includes at least one high porosity layer.

5. The sensor according to claim 4, wherein the at least one high refractive index layer includes at least one low porosity layer.

6. The sensor according to claim 2, wherein the at least one high refractive index layer includes at least one low porosity layer.

7. The sensor according to claim 1, wherein the at least one low refractive index layer includes at least one porous silicon layer.

8. The sensor according to claim 7, wherein the at least one low refractive index layer includes at least one high porosity layer.

9. The sensor according to claim 1, further comprising at least one prism.

10. The sensor according to claim 9, wherein the at least one prism includes a rutile prism.

11. The sensor according to claim 1, further comprising a silicon substrate.

12. A method of detecting at least one target species, the method comprising:
binding the at least one target species to a waveguide layer;
coupling light through the waveguide layer, the waveguide layer including at least one porous silicon layer having greater porosity than at least one second porous silicon layer;
coupling light through the at least one second porous silicon layer; and
identifying the at least one target species based on at least one of the coupling steps.

13. The method according to claim 12, further comprising coupling light through at least one silicon substrate.

14. The method according to claim 12, further comprising adjusting a level of porosity of at least one of the first porous silicon layer and the second porous silicon layer.

15. The method according to claim 14, wherein the adjusting is based at least in part on at least one characteristic of the at least one target molecule.

16. The method according to claim 15, wherein the at least one characteristic includes a size of the target molecule.

17. A system for detecting at least one target species, the system comprising:
means for binding the at least one target species to a waveguide layer;
means for coupling light through the waveguide layer; and
means for identifying the at least one target species.

18. The system according to claim 17, wherein the waveguide layer includes at least a first porous silicon layer and a second porous silicon layer.

19. The system according to claim 18, wherein the first porous silicon layer has a higher porosity than the second porous silicon layer.

20. The system according to claim 18, wherein a porosity of at least one of the first porous silicon layer and the second porous silicon layer is adjusted based at least in part on at least one characteristic of the at least one target species.

21. The system according to claim 20, wherein the at least one characteristic includes a size of the target species.
